(19) United States
(12) Patent Application Publication
(10) Pub. No.: US 2020/0085941 A1
(43) Pub. Date: Mar. 19, 2020

(54) ANTIGENIC THERMOSTABLE POLIO VACCINES & RELATED METHODS

(71) Applicant: Universal Stabilization Technologies, Inc., San Diego, CA (US)

(72) Inventor: Victor Bronshtein, San Diego, CA (US)

(73) Assignee: Universal Stabilization Technologies, Inc., San Diego, CA (US)

(21) Appl. No.: 16/609,200

(22) PCT Filed: Apr. 30, 2018
(86) PCT No.: PCT/US18/30307

§ 371 Date: Oct. 28, 2019

Related U.S. Application Data
(60) Provisional application No. 62/491,907, filed on Apr. 28, 2017.

(51) Int. Cl. A61K 39/13; C12N 7/00; A61K 9/16; A61K 47/02; A61K 47/18; A61K 47/26

(52) U.S. Cl. CPC A61K 39/13; C12N 7/00; A61K 9/1611; A61K 9/1617; A61K 9/1623; A61K 2039/5252; A61K 47/183; A61K 47/26; C12N 2770/32651; C12N 2770/32661; A61K 47/02

(57) ABSTRACT

The disclosure concerns vaccines; and more particularly, highly antigenic thermostable vaccines configured for mucosal or transdermal delivery without reconstitution. Also disclosed are methods for formulating the highly antigenic thermostable vaccines.

### TECHNICAL FIELD

This invention relates to vaccines; and more particularly, to highly antigenic thermostable vaccines configured for mucosal or transdermal delivery without reconstitution.

### SUMMARY OF INVENTION

### Technical Problem

There is a need for thermostable viral vaccines which are deliverable, without reconstitution, via mucosal or transdermal routes.

Viral vaccines, including rabies (ERA333), measles, rubella, MVA, YF 17D, and many others, have been preserved using a process known as Preservation by Vaporization (PBV) as described in commonly owned U.S. Pat. No. 9,469,835, issued Oct. 18, 2016. In the prior art, these viral vaccines are generally mixed in a preservation mixture, and subsequently dried using the PBV process.

However, simply states, it has been discovered that conventional preservation mixtures and techniques are not easily applied to poliovirus. After attempting to formulate poliovirus vaccines using conventional processes, such as PBV, the polio vaccines failed to achieve, inter alia, adequate stability.

For example, anhydrous micronized poliovirus vaccines are desired for mucosal or transdermal delivery. However, it has been discovered that such anhydrous micronized poliovirus vaccines prepared using a conventional preservation process are less stable at higher ambient temperatures. Stability of vaccines is an important component which affects the usefulness and efficacy of these products.

### Solution to Problem

It was hypothesized that the vaccine needed additional protection during the preservation process, and perhaps the inactivation process. In order to adequately protect vaccine components during the preservation process and to yield thermostable vaccine product achieving the desired stability and efficacy, a number of experiments were performed.

It was discovered that poliovirus vaccines, particularly those in an anhydrous micronized form suitable for mucosal or transdermal delivery, could be effectively stabilized at higher ambient temperatures by immobilizing the poliovirus in novel protective compositions.

Moreover, the protected and immobilized poliovirus achieved improved stability and antigenicity subsequent to inactivation by irradiation (although subsequent irradiation is not required in some embodiments).

For example, our research identified that successful stabilization of poliovirus was achieved only with preservation solutions (preservation mixtures) comprising high concentrations of protective salts that have low water activity in saturated solutions (i.e.; water activity less than 50% in saturated solution). However, the protective salts by themselves did not sufficiently stabilize the virus, in fact, the importance of other components was surprisingly discovered. In particular, in addition to protective salts the preservation mixture also required amino acids and carbohydrates in order to achieve adequate stabilization and efficacy.

Proposed herein is an antigenic thermostable vaccine composition containing poliovirus, comprising in parts by weight: 10-20 parts of one or more protective salts, wherein said one or more protective salts are selected from those exhibiting less than 50% water activity in saturated solution; 5-20 parts of one or more amino acids, and 10-40 parts of one or more carbohydrates; wherein a combination of the protective salts, amino acids and carbohydrates forms an anhydrous glassy matrix, and wherein the poliovirus is immobilized in the anhydrous glassy matrix.

Also proposed is a method for formulation of the antigenic thermostable vaccine composition, the method comprises: immobilizing the poliovirus in the anhydrous glassy matrix, the immobilizing including: combining the poliovirus with an aqueous preservation mixture, the aqueous preservation mixture comprising in parts by weight: 10-20 parts of the one or more protective salts; 5-20 parts of the one or more amino acids, and 10-40 parts of the one or more carbohydrates; and vacuum drying the combined poliovirus, protective salts, amino acids, and carbohydrates for at least 6 hours at a temperature greater than or equal to 40° C.

Further details of the aspects and embodiments of the invention are provided in the appended detailed descriptions and incorporate the level of ordinary knowledge and skill in the art.

### Advantageous Effects of Invention

It has been discovered that a composition comprising a high concentration of protective salts having low water activity (less than 50% in saturated solution), with further combination of amino acids and carbohydrates, as disclosed herein, is useful to preserve poliovirus and achieve an anhydrous glassy matrix containing the poliovirus and protective components. The dried glassy matrix is useful to immobilize a viral vaccine for protecting the vaccine during the stability preservation process, and perhaps also an inactivation by irradiation process. In some embodiments, subsequent inactivation by irradiation (for inactivating the viral vaccine) does not destroy useful proteins and epitopes of the viral vaccine because of the protective salts, amino acids and carbohydrates immobilizing the viral particles of the vaccine.

The compositions and methods disclosed herein provide a viral vaccine embedded in a micronized powder which can be implemented in a thermostable vaccine product. The thermostable vaccine product does not require cold-chain storage and can be delivered by mucosal or transdermal routes, without reconstitution.

Activity loss is improved, thereby providing improved vaccine efficacy of the disclosed compositions.

While the composition described herein was discovered in the context of studies with poliovirus, it is reasonable to expect that other viruses may be similarly protected using similar compositions to achieve similar results.

Other advantageous effects are hereinafter described.

### DESCRIPTION OF EMBODIMENTS

Commonly-owned U.S. Pat. No. 9,744,227, issued Aug. 29, 2017, and titled “COMPOSITIONS CONTAINING AMBIENT-TEMPERATURE STABLE, INACTIVATED BUT THERAPEUTICALLY ACTIVE BIOPHARMACEUTICALS & METHODS FOR FORMULATION THEREOF” describes a two-step approach for production of a thermostable, killed vaccines with high antigenicity, the two steps include:

Step 1: formulation of a thermostable live attenuated vaccine (LAV) using a process known as Preservation by Vaporization (PBV) as described in commonly owned U.S. Pat. No. 9,469,835, issued Oct. 18, 2016; and

Step 2. inactivation by irradiating the PBV-preserved LAV with an energy dose above 12.5 kGy, wherein high energy radiation selectively inactivates internal components without damaging antigens which are stabilized in a carbohydrate matrix.

The entire contents of these properties: U.S. Pat. No. 9,744,227 and U.S. Pat. No. 9,469,835 are hereby incorporated by reference.

The above-described two step approach has been successfully applied for development of inactivated thermostable vaccines against anthrax, listeria, and rabies. However, as the below data indicates, conventional preservation mixtures combined with PBV-preservation methods have failed to yield highly antigenic thermostable poliovirus for mucosal or transdermal delivery without reconstitution.

It was hypothesized by the named-inventor that added protections of important proteins and epitopes may be required to improve stability and efficacy of the resulting vaccine. As such, various compositions were investigated with the PBV process and subsequent inactivation process as described above, which led to a surprising observation that a combination of protective salts, amino acids and carbohydrates were effective in protecting viral vaccine during the process and thereby providing an improved viral vaccine composition for mucosal and transdermal delivery, as well as novel methods for formulating the vaccine.

For purposes herein, thermostable vaccines are vaccines that are stable for at least ninety days at all ambient temperatures (AT) between −20° C. to +37 ° C.

For purposes herein, high ambient temperatures is a relative term which includes those ambient temperatures greater than 27° C.

Now, in accordance with one aspect, herein disclosed is an antigenic thermostable vaccine composition containing virus. The composition comprises in parts by weight: (i) 10-20 parts of one or more protective salts, wherein said one or more protective salts are selected from those exhibiting less than 50% water activity in saturated solution; (ii) 5-20 parts of one or more amino acids, and (iii) 10-40 parts of one or more carbohydrates; wherein a combination of the protective salts, amino acids and carbohydrates forms an anhydrous glassy matrix, and wherein the virus is immobilized in the anhydrous glassy matrix.

In an embodiment, the virus may comprise Sabin live attenuated polio vaccine. In another embodiment, the virus may comprise inactivated polio virus. IN yet other embodiments the virus may be other than poliovirus.

In an embodiment, the one or more protective salts may comprise: magnesium chloride, potassium acetate, or a combination thereof. Other similar salts may be experimentally validated and implemented without undue experimentation. Alternatively, other similar salts may be recognized and implemented within the scope of the knowledge and skill generally possessed by one having skill in the art.

In an embodiment, the one or more amino acids may comprise: glutamic acid, glycine, proline, serine, threonine, valine, arginine, alanine, lysine, cysteine, or any salt or combination thereof. Other similar amino acids may be experimentally validated and implemented without undue experimentation. Alternatively, other similar amino acids may be recognized and implemented within the scope of the knowledge and skill generally possessed by one having skill in the art.

In an embodiment, the one or more carbohydrates may comprise one or more monosaccharides, oligosaccharides, sugar alcohols, or a combination thereof. The one or more monosaccharides may comprise methyl glucoside. The one or more oligosaccharides may comprise sucrose, maltose, trehalose, lactose, meibiose, cellobiose, or a combination thereof. The one or more sugar alcohols may comprise sorbitol, mannitol, glycerol, lactitol, dulcitol, xylitol, erythritol, isomalt, or a combination thereof. Other similar carbohydrates may be experimentally validated and implemented without undue experimentation. Alternatively, other similar carbohydrates may be recognized and implemented within the scope of the knowledge and skill generally possessed by one having skill in the art.

In an embodiment, the anhydrous glassy matrix may comprise a plurality of micronized particles, wherein the micronized particles are configured for mucosal or transdermal delivery without reconstitution.

In one embodiment, each of the micronized particles comprise a diameter less than or equal to 50 micrometers. In another embodiment, each of the micronized particles diameter less than or equal to 40 micrometers. In another embodiment, each of the micronized particles diameter less than or equal to 30 micrometers. In another embodiment, each of the micronized particles diameter less than or equal to 20 micrometers. In another embodiment, each of the micronized particles diameter less than or equal to 5 micrometers.

In another aspect, a method for formulating the antigenic thermostable vaccine composition containing virus comprises: (i) immobilizing the virus in said anhydrous glassy matrix, said immobilizing including: combining the virus with an aqueous preservation mixture, the aqueous preservation mixture comprising in parts by weight: 10-20 parts of said one or more protective salts; 5-20 parts of said one or more amino acids, and 10-40 parts of said one or more carbohydrates; and (ii) drying the combined virus, protective salts, amino acids, and carbohydrates, wherein said drying comprises vacuum drying for at least 6 hours at a temperature greater than or equal to 40° C.

In an embodiment, the method further comprises: subsequent to immobilizing the virus in the anhydrous glassy matrix, inactivating the virus by irradiating said anhydrous glassy matrix containing the virus using a permeated ionizing radiation dose above 12.5 kGy. The permeated ionizing radiation dose may be delivered by: electron beam irradiation, gamma irradiation, or X-ray irradiation.

In another embodiment, the virus is inactivated prior to said combining the poliovirus with an aqueous preservation mixture.

The combination of protective salts, amino acids, and carbohydrates function to protect the virus during preservation (stabilization). These components of the composition are also protective of the virus during an optional post-preservation inactivation step, such as inactivation by irradiation.

Now, in order to further illustrate the various aspects and embodiments of the invention, certain non-limiting illustrative examples are provided such that one with skill in the art may further appreciate and be enabled to make and use the invention as-claimed.

### Example 1—Problem

Oral polio vaccine (OPV) was preserved using Preservation by Vaporization (PBV). Activity of oral polio vaccine (OPV) before and after PBV dying was quantified using a standard endpoint dilution assay to measure 50% cell culture infective dose (CCID50) on human rhabdomyosarcoma (RD) cells from ATCC (ATCC® CCL-136™). RD cells were infected with serial dilutions of reconstituted PBV polio samples and frozen control samples, and each underwent incubation for 5 days at 33° C. Cytopathic effect (CPE) was then observed under magnification and final results were calculated using the Spearman-Karber method.

Before drying, the OPV suspension was mixed with preservation mixtures (PM) including ingredients that had been previously used to successfully stabilize many viral vaccines including rabies (ERA333), measles, rubella, MVA, YF 17D, and many others. The results are in the Table 1 below.

TABLE 1
Activity of OPV after PBV

| | OPV activity loss |
|---|---|
| Composition of Preservation Mixture | after PBV (logs) |
| sucrose 30%, mannitol 10% | −2.96 |
| sucrose 30%, mannitol 10%, 0.5% gelatin | −2.04 |
| sucrose 30%, methyl glucoside 10% | −2.71 |
| sucrose 30%, methyl glucoside 10%, 0.5% | −2.88 |
| gelatin | |

The results as shown in Table 1 show that conventional formulations of preservation mixtures do not achieve useful stability for OPV.

### Example 2—Discovery

In another experiment, oral polio vaccine (OPV) was preserved using Preservation by Vaporization (PBV). Before drying, OPV suspension was mixed with preservation mixtures as shown in Table 2, below. OPV activity loss is illustrated after completing PBV, after 45 days (1.5 months) at room temperature (RT), after 45 days at 37° C., after 165 days (5.5 months) at RT, and after 165 days at 37° C.; the results are indicated in Table 2.

TABLE 2
Activity of OPV after PBV and subsequent storage at RT and 37° C.

| | OPV activity loss (logs) | | | | |
|---|---|---|---|---|---|
| | | After 1.5 | After 1.5 | After 5.5 | After 5.5 |
| Composition of | After | months at | months at | months at | months at |
| Preservation Mixture | PBV | RT | 37° C. | RT | 37° C. |
| sucrose 20%, mannitol 10%, | −0.54 | −1.35 | −1.52 | −1.00 | −2.67 |
| MgCl2 10%, MSG 10% | | | | | |
| sucrose 15%, mannitol 10%, | −0.79 | −1.02 | −1.68 | −1.18 | −2.42 |
| MgCl2 10%, MSG 15% | | | | | |
| sucrose 20%, mannitol 10%, | −1.42 | −1.14 | −1.89 | −1.48 | −2.58 |
| MgCl2 5%, MSG 10% | | | | | |
| sucrose 15%, mannitol 10%, | −0.92 | −1.64 | −1.97 | −1.83 | −2.58 |
| MgCl2 5%, MSG 15% | | | | | |
| sucrose 20%, sorbitol 10%, | −1.04 | −1.77 | −1.77 | −1.33 | −2.58 |
| MgCl2 10%, MSG 10% | | | | | |
| sucrose 15%, sorbitol 10%, | −0.54 | −0.60 | −1.43 | −1.42 | −2.58 |
| MgCl2 10%, MSG 15% | | | | | |

The results as shown in Table 2 indicate that preservation mixtures comprising MgCl2 and MSG better protect OPV activity after drying compared to conventional PBV preservation mixtures (see Table 1); but there remains a need for further improving stability of preserved OPV at ambient temperatures.

The experimental results further indicate an improvement when using a preservation mixture which comprises (i) protective salts (ex: MgCl2), (ii) amino acids (ex: monosodium glutamate or “MSG”), and (iii) carbohydrates (ex: sucrose, sorbitol).

### Example 3—Effects of Freezing

The effects of freezing the OPV prior to drying on its activity were evaluated. Frozen and never frozen oral polio vaccine (OPV) were preserved using Preservation by Vaporization (PBV). Before drying, OPV suspensions were mixed with preservation mixtures as shown in Table 3, below.

TABLE 3
Activity of frozen and never frozen OPV after PBV and subsequent storage at RT and
37° C.

| | OPV activity loss (logs) | | | |
|---|---|---|---|---|
| | | After 2 | After 2 | After 4 |
| | | months at | months at | months at |
| Composition of Preservation Mixture | After PBV | RT | 37° C. | RT |

OPV was frozen to-80° C. prior to drying

| | | | | |
|---|---|---|---|---|
| PM1-frozen13.2% sorbitol + 17% MgCl2 + | −0.64 | N/A | −1.97 | −0.22 |
| 17% MSG | | | | |
| PM2-frozen13.2% sorbitol + 17% | −0.56 | N/A | −1.89 | −0.47 |
| potassium acetate + 17% MSG | | | | |

OPV was never frozen

| | | | | |
|---|---|---|---|---|
| PM1-unfrozen13.2% sorbitol + 17% MgCl2 + | −0.89 | −0.56 | −1.64 | −0.14 |
| 17% MSG | | | | |
| PM2-unfrozen13.2% sorbitol + 17% | −0.47 | −0.64 | −2.06 | −0.47 |
| potassium acetate + 17% MSG | | | | |

As shown in Table 3, no negative effect of freezing before drying was identified. The preservation mixtures containing protective salts, amino acids and carbohydrates yielded high stability at RT, as shown.

### Example 4: Activity and Antigenicity of OPV in Preservation Mixtures

OPV was preserved using Preservation by Vaporization (PBV). Before drying, OPV suspension was mixed with preservation mixtures as shown in Tables 4 and 5, below. In this experiment we compared stability of OPV activity and antigenicity, respectively, after 1-month storage at 37° C. The antigenicity was tested using a direct sandwich ELISA to detect D-antigen unites content. The assay uses rabbit polyclonal serotype-1-specific IgG, detected using Biotin-conjugated rabbit IgG, followed by ExtrAvidin-peroxidase conjugate. A color reaction is developed using tetra methyl benzidine (TMB) and stopped with an acid fixative solution. ELISA plates are scanned by microplate reader at 450 nm to quantify response from serial dilutions of reference and test samples. D-antigen content was determined by statistical analysis using linear regression of optical density (OD) data.

TABLE 4
Activity of OPV after PBV and subsequent 1-month storage at 37° C.

| | | OPV activity |
|---|---|---|
| Composition of Preservation | OPV activity loss | loss after 1 month |
| Mixture | after PBV (logs) | 37° C. (logs) |
| sorbitol 13.2%, MgCl2 17%, MSG | −0.81 | −1.98 |
| 17% | | |
| methyl-a-glucoside (MAG) 13.2%, | −0.64 | −1.81 |
| potassium acetate 17%, MSG 17% | | |
| sorbitol 13.2%, potassium acetate | −0.48 | −1.48 |
| 17%, MSG 17% | | |

TABLE 5
Antigenicity of OPV after PBV and
subsequent 1-month storage at 37° C.

| | OPV | OPV Antigenicity |
|---|---|---|
| Composition of Preservation | Antigenicity loss | loss after 1 month |
| Mixture | after PBV (logs) | at 37° C. (logs) |
| sorbitol 13.2%, MgCl2 17%, MSG | 0.09 log loss | 0.15 log loss |
| 17% | | |
| methyl-a-glucoside (MAG) 13.2%, | 0.26 log loss | 0.26 log loss |
| potassium acetate 17%, MSG 17% | | |
| sorbitol 13.2%, potassium acetate | 0.37 log loss | 0.26 log loss |
| 17%, MSG 17% | | |

The results as shown in Tables 4-5 indicate that activity and antigenicity are preserved using the example preservation mixtures as disclosed. Similar results were recognized using other compositions comprising in parts by weight: 10-20 parts of one or more protective salts, wherein said one or more protective salts are selected from those exhibiting less than 50% water activity in saturated solution; 5-20 parts of one or more amino acids, and 10-40 parts of one or more carbohydrates.

### Example 5—Activity and Antigenicity of OPV in Preservation Mixtures After Irradiation

In these experiments, the same preservations mixtures of Example 4 were selected for inactivation by electron beam irradiation to produce Sabin inactivated polio vaccine (SIPV).

TABLE 6
Activity of PBV preserved OPV after irradiation (CCID50/ml)

| | | | Composition of |
|---|---|---|---|
| Radiation doze | OPV activity | Logs of loss | Preservation Mixture |
| 6 kGy | 7.64 E4 | −2.95 | sorbitol 13.2%, MgCl2 |
| 12 kGy | 4.00 E3 | −4.23 | 17%, MSG 17% |
| 6 kGy | 2.00 E4 | −3.53 | |
| 12 kGy | 1.37 E3 | −4.70 | MAG 13.2%, potassium |
| | | | acetate 17%, MSG 17% |
| 6 kGy | 9.99 E4 | −2.83 | sorbitol 13.2%, |
| 12 kGy | 3.06 E3 | −4.35 | potassium acetate 17%, |
| | | | MSG 17% |

Frozen OPV control: 6.81 E7 (CCID50/ml)

TABLE 7
Loss of OPV Antigenicity after PBV and subsequent irradiation

| | OPV Antigenicity | After | After |
|---|---|---|---|
| | loss after PBV | subsequent | subsequent |
| Formulation | (logs) before | irradiation | irradiation |
| ingredients of PS | irradiation | at 6 kGy | at 12 kGy |
| sorbitol 13.2%, MgCl2 | 0.09 log loss | 0.16 log loss | 0.27 log loss |
| 17%, MSG 17%, | | | |
| Methyl glucoside13.2%, | 0.26 log loss | 0.32 log loss | 0.56 log loss |
| potassium acetate 17%, | | | |
| MSG 17% | | | |
| sorbitol 13.2%, | 0.37 log loss | 0.28 log loss | 0.60 log loss |
| potassium acetate 17%, | | | |
| MSG 17% | | | |

The results as shown in Tables 6-7 indicate that activity and antigenicity post-irradiation are preserved using the example preservation mixtures as disclosed. Similar results were recognized using other compositions comprising in parts by weight: 10-20 parts of one or more protective salts, wherein said one or more protective salts are selected from those exhibiting less than 50% water activity in saturated solution; 5-20 parts of one or more amino acids, and 10-40 parts of one or more carbohydrates.

Where percentages are used, it should be understood that the percentages are percent by weight (%/w).

Because the invention describes aqueous solutions and drying (vaporization or otherwise removing water to form an anhydrous glassy matrix), it is best to consider the preservation mixtures in terms of parts by weight, since, as water vaporized the ratio of parts remains the same while percent of the aggregate product varies with respect to water content. For this reason, the compositions of preservations mixtures may be better understood in terms of parts by weight without regard to water content.

Now, while various preservation mixtures are explicitly described in the above examples, it is to be understood that other compositions for preservation mixtures are further contemplated but are too numerous to list with particular reference.

We have identified in the above description a number of protective salts, amino acids and carbohydrates which are useful for preparing a preservation mixture and implementing with embodiments and aspects of the claimed-invention. However, other similar components may be utilized which are expected to provide similar results.

As such, it should be recognized that the preservation mixtures, and resulting compositions for viral vaccines, may comprise a number of alternatives of protective salts, amino acids, and carbohydrates which are not described in the examples. One having skill in the art, along with the conventional knowledge in the art, and the description herein, will be adequately enabled to make and use the claimed invention without undue experimentation. As such, the above-described examples are intended to be non-limiting of the spirit and scope of the claimed invention.

### INDUSTRIAL APPLICABILITY

The disclosure concerns highly antigenic thermostable vaccines for mucosal or transdermal delivery without reconstitution, and methods for formulation thereof. These vaccines are generally applicable to the medical field.

### CITATION LIST

U.S. Pat. No. 9,469,835, issued Oct. 18, 2016.

U.S. Pat. No. 9,744,227, issued Aug. 29, 2017.

What is claimed is:

1. An antigenic thermostable vaccine composition containing virus, comprising in parts by weight:
10-20 parts of one or more potassium salts, wherein said one or more potassium salts are selected from those exhibiting less than 50% water activity in saturated solution;
5-20 parts of one or more amino acids, and
10-40 parts of one or more carbohydrates;
wherein a combination of the potassium salts, amino acids and carbohydrates forms an anhydrous glassy matrix, and
wherein the virus is immobilized in the anhydrous glassy matrix.

2. The vaccine composition of claim 1, wherein said virus further comprises Sabin live attenuated polio vaccine.

3. The vaccine composition of claim 1, wherein said virus comprises inactivated polio virus.

4. The vaccine composition of claim 1, wherein said one or more potassium salts comprises potassium acetate.

5. The vaccine composition of claim 1, wherein said one or more amino acids comprises: glutamic acid, glycine, monosodium glutamate, proline, serine, threonine, valine, arginine, alanine, lysine, cysteine, or any salt or combination thereof.

6. The vaccine composition of claim 1, wherein said one or more carbohydrates comprises one or more monosaccharides, oligosaccharides, sugar alcohols, or a combination thereof.

7. The vaccine composition of claim 6, wherein said one or more monosaccharides comprises: methyl glucoside.

8. The vaccine composition of claim 6, wherein said one or more oligosaccharides comprises: sucrose, maltose, trehalose, lactose, meibiose, cellobiose, or a combination thereof.

9. The vaccine composition of claim 6, wherein said one or more sugar alcohols comprises sorbitol, mannitol, glycerol, lactitol, dulcitol, xylitol, erythritol, isomalt, or a combination thereof.

10. The vaccine composition of claim 1, wherein the anhydrous glassy matrix comprises a plurality of micronized particles, wherein said micronized particles are configured for mucosal or transdermal delivery without reconstitution.

11. The vaccine composition of claim 8, wherein each of the micronized particles comprise a diameter less than or equal to 50 micrometers.

12. The vaccine composition of claim 8, wherein each of the micronized particles diameter less than or equal to 40 micrometers.

13. The vaccine composition of claim 8, wherein each of the micronized particles diameter less than or equal to 30 micrometers.

14. The vaccine composition of claim 8, wherein each of the micronized particles diameter less than or equal to 20 micrometers.

15. The vaccine composition of claim 8, wherein each of the micronized particles diameter less than or equal to 5 micrometers.

16. A method for formulating the composition of claim 1, comprising:
immobilizing the virus in said anhydrous glassy matrix, said immobilizing including:
combining the virus with an aqueous preservation mixture, the aqueous preservation mixture comprising in parts by weight:
10-20 parts of said one or more potassium salts;
5-20 parts of said one or more amino acids, and
10-40 parts of said one or more carbohydrates; and
drying the combined virus, potassium salts, amino acids, and carbohydrates, wherein said drying comprises vacuum drying for at least 6 hours at a temperature greater than or equal to 40° C.

17. The method of claim 16, further comprising:
subsequent to immobilizing the poliovirus in the anhydrous glassy matrix:
inactivating the poliovirus by irradiating said anhydrous glassy matrix containing the poliovirus using a permeated ionizing radiation dose above 12.5 kGy.

18. The method of claim 17, wherein the permeated ionizing radiation dose is delivered by: electron beam irradiation, gamma irradiation, or X-ray irradiation.

19. The method of claim 16, wherein the poliovirus is inactivated prior to said combining the poliovirus with an aqueous preservation mixture.
